(19) United States
(12) Patent Application Publication
(10) Pub. No.: US 2024/0079084 A1
(43) Pub. Date: Mar. 7, 2024

(54) METHODS, SYSTEMS, AND MEDIA METHOD APPLYING MACHINE LEARNING TO CHEMICAL MAPPING DATA FOR RNA TERTIARY STRUCTURE PREDICTION

(71) Applicant: Atomic AI, Inc., South San Francisco, CA (US)

(72) Inventors: Raphael John Lamarre TOWNSHEND, Menlo Park, CA (US); Stephan Johannes EISMANN, Redwood City, CA (US)

(21) Appl. No.: 18/452,480

(22) Filed: Aug. 18, 2023

Related U.S. Application Data
(63) Continuation of application No. PCT/US2023/030635, filed on Aug. 18, 2023.
(60) Provisional application No. 63/371,983, filed on Aug. 19, 2022.

(51) Int. Cl. G16B 15/10; G16B 45/00

(52) U.S. Cl. CPC G16B 15/10; G16B 45/00

(57) ABSTRACT

Disclosed herein are methods, systems, and media for predicting a tertiary structure of a target RNA molecule comprising: creating a training data set comprising chemical mapping data for one or more of a first plurality of RNA molecules and tertiary structure data for one or more of a second plurality of RNA molecules; training a machine learning algorithm using the training data set; applying the trained machine learning algorithm to predict the tertiary structure of an the RNA molecule of interest; and outputting the predicted tertiary structure of the RNA molecule of interest.

### CROSS-REFERENCE

This application is a continuation of International Application No. PCT/US2023/030635, filed Aug. 18, 2023, which claims the benefit of U.S. Provisional Application No. 63/371,983, filed on Aug. 19, 2022, each of which are incorporated by reference in their entirety.

### BACKGROUND

The 3D structure of biological molecules plays a crucial role in determining their function. Nucleic acids, such as RNA molecules, are one class of biological molecules. Despite this, experimental determination of RNA tertiary structure through crystallography-based diffraction imaging, electron microscopy or nuclear magnetic resonance spectroscopy is costly and slow. In addition, due to the flexibility of RNA molecules, it is often not possible to determine their structure using these experimental methods. Thus, there remains an urgent need for an efficient and inexpensive way to determine the structures of RNA molecules.

### SUMMARY

In some aspects, the present disclosure provides a computer-implemented method of predicting a tertiary structure of an RNA molecule of interest comprising: creating a training data set comprising: chemical mapping data for a first plurality of RNA molecules, and tertiary structure data for a second plurality of RNA molecules; training a machine learning algorithm using the training data set; applying the trained machine learning algorithm to predict the tertiary structure of the RNA molecule of interest; and outputting the predicted tertiary structure of the RNA molecule of interest.

In some aspects, the present disclosure provides a computer-implemented method of predicting tertiary structure of an RNA molecule of interest comprising: obtaining a machine-learning model, wherein the machine-learning model was trained by a process including: creating a training data set comprising chemical mapping data for a first plurality of RNA molecules and tertiary structure data for a second plurality of RNA molecules; and training the machine-learning model using the training data set; applying the machine-learning model to predict the tertiary structure of the RNA molecule of interest; and outputting the predicted tertiary structure of the RNA molecule of interest.

In some embodiments, the chemical mapping data is generated by a process comprising contacting the RNA molecule with a chemical probing agent, optionally wherein the RNA molecule is at least one of the first plurality of RNA molecules or the RNA molecule of interest.

In some embodiments, the chemical probing agent comprises dimethyl sulfate (DMS).

In some embodiments, the chemical probing agent comprises a SHAPE (selective 2′-hydroxyl acylation and primer extension) reagent.

In some embodiments, the SHAPE reagent is 1-methyl-7-nitroisatoic anhydride (1M7), 1-methyl-6-nitroisatoic anhydride (1M6), 5-nitroisatoic anhydride (5NIA), or N-methyl-nitroisatoic anhydride (NMIA).

In some embodiments, the chemical probing agent comprises 2A3 ((2-Aminopyridin-3-yl)(1H-imidazol-1-yl)methanone).

In some embodiments, the RNA molecule of interest comprises a part of a transcriptome.

In some embodiments, the transcriptome is a human transcriptome.

In some embodiments, the training set comprises chemical mapping data for at least about 10, 100, 500, 1,000, 10,000, or more than 10,000 sequences.

In some embodiments, the training set comprises chemical mapping data for at most about 10, 100, 500, 1,000, or 10,000 sequences.

In some embodiments, the chemical mapping data is for sequences which occur in different abundance than in natural systems.

In some embodiments, the chemical mapping data was collected from in vitro sources.

In some embodiments, the method further comprises, before applying the machine-learning model, tuning the machine learning algorithm based on a chemical mapping data of the RNA molecule of interest.

In some embodiments, the machine learning algorithm comprises one or more artificial neural networks (ANNs).

In some embodiments, the method further comprises training the ANN to predict chemical mapping data for the RNA molecule of interest.

In some embodiments, the method further comprises predicting the chemical mapping data for the RNA molecule of interest from the predicted tertiary structure of the RNA molecule of interest.

In some embodiments, the method further comprises predicting the predicted tertiary structure of the RNA molecule of interest based on the chemical mapping data for the RNA molecule of interest.

In some embodiments, the method further comprises predicting the chemical mapping data for the RNA molecule of interest and the predicted tertiary structure of the RNA molecule of interest using the same embedding.

In some embodiments, the tertiary structure comprises 3-D coordinates of a plurality of atoms that compose the RNA molecule of interest.

In some embodiments, the tertiary structure comprises 3-D coordinates of each atom that composes the RNA molecule of interest.

In some embodiments, the tertiary structure comprises one or more 3-D coordinates for a plurality of nucleotides that compose the RNA molecule of interest.

In some embodiments, the tertiary structure comprises one or more 3-D coordinates for each nucleotide that composes the RNA molecule of interest.

In some embodiments, the tertiary structure of the RNA molecule of interest is parametrized based on a distance map.

In some embodiments, the tertiary structure of the RNA molecule of interest is parametrized based on a distance map and angles.

In some embodiments, the method does not require determining or predicting a secondary structure of the RNA molecule of interest.

In some embodiments, the method predicts aspects of a tertiary structure of the target RNA that are not captured by a base-pairing prediction of the target RNA.

In some embodiments, the predicted tertiary structure comprises one or more of a pseudoknot, multi-way junction, coaxial stack, a-minor motif, kissing stem-loop, ribose zipper, or tetraloop/tetraloop receptor.

In some embodiments, the chemical mapping data comprises multidimensional chemical mapping data for one or more RNA molecules of the first plurality of RNA molecules.

In some embodiments, the predicted tertiary structure is a target for a pharmaceutical drug.

In some embodiments, the method further comprises determining a target region or subsequence of the RNA molecule of interest, for a pharmaceutical drug to target, based on the predicted tertiary structure.

In some embodiments, the method further comprises formulating a pharmaceutical drug based on the predicted tertiary structure.

In some embodiments, the training data set further comprises a multiple sequence alignment of a third plurality of RNA molecules.

In some embodiments, the first plurality of RNA molecules and the second plurality of RNA molecules are the same or different.

In some embodiments, the first plurality of RNA molecules and the third plurality of molecules are the same.

In some embodiments, the first plurality of RNA molecules are unrelated to the RNA molecule of interest.

In some embodiments, the second plurality of RNA molecules are unrelated to the RNA molecule of interest.

In some embodiments, the third plurality of RNA molecules are unrelated to the RNA molecule of interest.

In some embodiments, an RNA molecule of the first plurality of RNA molecules has no more than about 80%, 70%, 60%, 50%, 40%, 30%, 20%, or less sequence identity to the RNA molecule of interest.

In some aspects, the present disclosure provides a computer-implemented system for predicting a tertiary structure of an RNA molecule of interest comprising a computing device comprising at least one processor and instructions executable by the at least one processor to perform operations comprising: creating a training data set comprising one or more of: chemical mapping data for a first plurality of RNA molecules, tertiary structure data for a second plurality of RNA molecules; training a machine learning algorithm using the training data set; applying the trained machine learning algorithm to predict the tertiary structure of the RNA molecule of interest; and outputting the predicted tertiary structure of the RNA molecule of interest.

In some aspects, the present disclosure provides a non-transitory computer-readable storage media encoded with instructions executable by one or more processors to provide an application for predicting a tertiary structure of an RNA molecule of interest, the application comprising: a training data set module configured to create a training data set comprising: chemical mapping data for a first plurality of RNA molecules, and tertiary structure data for a second plurality of RNA molecules; a training module configured to train a machine learning algorithm using the training data set; an inference module configured to apply the trained machine learning algorithm to predict the tertiary structure of the RNA molecule of interest; and an output module configured to report the predicted tertiary structure of the RNA molecule of interest.

In some aspects, the present disclosure provides a non-transitory computer-readable medium comprising: accessing an RNA tertiary structure prediction system that was manufactured by a process comprising: creating a training data set comprising chemical mapping data for a first plurality of RNA molecules and tertiary structure data for a second plurality of RNA molecules; training a machine-learning model using the training data set; and storing the trained machine-learning model on the non-transitory computer-readable medium; and computer program code that, when executed by a computing system, cause the computing system to perform operations including: using the RNA tertiary structure prediction system to predict the tertiary structure of the RNA molecule of interest; and outputting the predicted tertiary structure of the RNA molecule of interest.

In some aspects, the present disclosure provides a computer-implemented method of predicting tertiary structure of an RNA molecule of interest comprising: sending a query for predicting the tertiary structure of the RNA molecule of interest to a computer comprising a machine-learning model, wherein the machine learning algorithm generates the tertiary structure, wherein the machine-learning model was trained by a process including: creating a training data set comprising chemical mapping data for a first plurality of RNA molecules and tertiary structure data for a second plurality of RNA molecules; and training the machine-learning model using the training data set; receiving the predicted tertiary structure of the RNA molecule of interest from the computer.

Accordingly, aspects of the present disclosure comprise methods and systems for RNA tertiary structure prediction by creating a training set that comprises chemical mapping data for one or more RNA molecules of a first plurality of RNA molecules and tertiary structure data for one or more RNA molecules of a second plurality of RNA molecules. The training data may optionally include other types of data such as multiple sequence alignments or predicted or observed secondary structures. The training set may include different types of data for each training item and the chemical mapping data may be from multiple experimental methods or using a variety of parameters. The training set may then be used for training the computational method that comprises a machine learning algorithm; and the trained machine learning algorithm may be used to predict the tertiary structure of one or more RNA molecules of interest; and then output the predicted tertiary structure of the RNA molecule(s) of interest.

An aspect of the present disclosure provides a computer-implemented method of predicting a tertiary structure of an RNA molecule of interest comprising: (a) creating a training data set comprising: (i) chemical mapping data for a first plurality of RNA molecules, and (ii) tertiary structure data for a second plurality of RNA molecules; (b) training a machine learning algorithm using the training data set; (c) applying the trained machine learning algorithm to predict the tertiary structure of the RNA molecule of interest; and (d) outputting the predicted tertiary structure of the RNA molecule of interest. In some embodiments, the chemical mapping data is generated by a process comprising contacting the RNA molecule with a chemical probing agent. In some embodiments, the chemical probing agent comprises dimethyl sulfate (DMS). In some embodiments, the chemical probing agent comprises a SHAPE (selective 2′-hydroxyl acylation and primer extension) reagent. In some embodiments, the SHAPE reagent is 1-methyl-7-nitroisatoic anhydride (1M7), 1-methyl-6-nitroisatoic anhydride (1M6), 5-nitroisatoic anhydride (5NIA), or N-methyl-nitroisatoic anhydride (NMIA). In some embodiments, the chemical probing agent comprises 2A3 ((2-Aminopyridin-3-yl)(1H-imidazol-1-yl)methanone). In some embodiments, the RNA of interest comprises a part of a transcriptome. In some embodiments, the transcriptome is a human transcriptome. In some embodiments, the training set comprises chemical mapping data for at least about 10, 100, 500, 1,000, 10,000, or more than 10,000 sequences. In some embodiments, the training set comprises chemical mapping data for at most about 10, 100, 500, 1,000, or 10,000 sequences. In some embodiments, the chemical mapping data is for sequences which occur in different abundance than in natural systems. In some embodiments, the chemical mapping data was collected from in vitro sources. In some embodiments, the machine learning algorithm comprises one or more artificial neural networks (ANNs). In some embodiments, the method further comprises training the ANN to predict chemical mapping data for the RNA of interest. In some embodiments, the method further comprises predicting the chemical mapping data for the RNA of interest from the predicted tertiary structure of the RNA of interest. In some embodiments, the method further comprises outputting features of the RNA molecule of interest generated by the machine learning algorithm. In some embodiments, the tertiary structure comprises 3-D coordinates of a plurality of atoms that compose the RNA molecule of interest. In some embodiments, the tertiary structure comprises 3-D coordinates of each atom that composes the RNA molecule of interest. In some embodiments, the tertiary structure comprises one or more 3-D coordinates for a plurality of nucleotides that compose the RNA molecule of interest. In some embodiments, the tertiary structure comprises one or more 3-D coordinates for each nucleotide that composes the RNA molecule of interest. In some embodiments, the tertiary structure of the RNA molecule of interest is parametrized based on a distance map. In some embodiments, the tertiary structure of the RNA molecule of interest is parametrized based on a distance map and angles. In some embodiments, the method does not require determining or predicting a secondary structure of the RNA molecule of interest. In some embodiments, the method predicts aspects of a tertiary structure of the target RNA that are not captured by a base-pairing prediction of the target RNA. In some embodiments, the predicted tertiary structure comprises one or more of a pseudoknot, multi-way junction, coaxial stack, a-minor motif, kissing stem-loop, ribose zipper, or tetraloop/tetraloop receptor. In some embodiments, the chemical mapping data comprises multidimensional chemical mapping data for one or more RNA molecules of the first plurality of RNA molecules. In some embodiments, the tertiary structure is a target for a pharmaceutical drug. In some embodiments, the training data set further comprises a multiple sequence alignment of a third plurality of RNA molecules. In some embodiments, the first plurality of RNA molecules and the second plurality of RNA molecules are the same. In some embodiments, the first plurality of RNA molecules and the third plurality of molecules are the same. In some embodiments, the first plurality of RNA molecules and the second plurality of RNA molecules are different. In some embodiments, the first plurality of RNA molecules and the third plurality of molecules are different. In some embodiments, the first plurality of RNA molecules and the second plurality of RNA molecules comprise mutually exclusive sets of RNA molecules. In some embodiments, the first plurality of RNA molecules and the third plurality of molecules comprise mutually exclusive sets of RNA molecules. In some embodiments, the first plurality of RNA molecules and the second plurality of RNA molecules comprise at least one different RNA molecule. In some embodiments, the first plurality of RNA molecules and the third plurality of molecules comprise at least one different RNA molecule. In some embodiments, the first plurality of RNA molecules and the second plurality of RNA molecules comprise at least one same RNA molecule. In some embodiments, the first plurality of RNA molecules and the third plurality of molecules comprise at least one same RNA molecule. In some embodiments, the first plurality of RNA molecules are unrelated to the RNA molecule of interest. In some embodiments, the second plurality of RNA molecules are unrelated to the RNA molecule of interest. In some embodiments, the third plurality of RNA molecules are unrelated to the RNA molecule of interest. In some embodiments, an RNA molecule of the first plurality of RNA molecules has no more than about 80%, 70%, 60%, 50%, 40%, 30%, 20%, or less sequence identity to the RNA molecule of interest.

Another aspect of the present disclosure provides a computer-implemented system for predicting a tertiary structure of an RNA molecule of interest comprising a computing device comprising at least one processor and instructions executable by the at least one processor to perform operations comprising: (a) creating a training data set comprising one or more of: (i) chemical mapping data for a first plurality of RNA molecules, (ii) structure data for a second plurality of RNA molecules; (b) training a machine learning algorithm using the training data set; (c) applying the trained machine learning algorithm to predict the tertiary structure of the RNA molecule of interest; and (d) outputting the predicted tertiary structure of the RNA molecule of interest.

Another aspect of the present disclosure provides a non-transitory computer-readable storage media encoded with instructions executable by one or more processors to provide an application for predicting a tertiary structure of an RNA molecule of interest, the application comprising: (a) a training data set module configured to create a training data set comprising: chemical mapping data for a first plurality of RNA molecules, and tertiary structure data for a second plurality of RNA molecules; (b) a training module configured to train a machine learning algorithm using the training data set; (c) an inference module configured to apply the trained machine learning algorithm to predict the tertiary structure of the RNA molecule of interest; and (d) an output module configured to report the predicted tertiary structure of the RNA molecule of interest.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a non-limiting example of a computing device; in this case, a device with one or more processors, memory, storage, and a network interface;

FIG. 2 shows a non-limiting example of a system for predicting RNA structure from sequence information;

FIG. 3A shows a non-limiting example of a system comprising machine learning algorithms described herein; and

FIGS. 3B and 3C show non-limiting examples of information flow during training of machine learning algorithms as comprised in systems and methods described herein.

### DETAILED DESCRIPTION

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein generally refers to a value approaching 100% of a given value. For example, a peptide that is “substantially localized” in an organ can indicate that about 90% by weight of a peptide, salt, or metabolite is present in an organ relative to a total amount of a peptide, salt, or metabolite. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “nucleic acid” generally refers to a polymeric form of nucleotides of any length, either deoxyribonucleotides or ribonucleotides, or analogs thereof, either in single-, double-, or multi-stranded form. A nucleic acid may be exogenous or endogenous to a cell. A nucleic acid may exist in a cell-free environment. A nucleic acid may be a gene or fragment thereof. A nucleic acid may be DNA. A nucleic acid may be RNA. A nucleic acid may have any three-dimensional structure and may perform any function. A nucleic acid may comprise one or more analogs (e.g., altered backbone, sugar, or nucleobase).

“In vitro” is defined as biological processes, reactions or experiments that are made to occur in isolation away from the whole organism, for example in a test tube, an artificial environment or in culture. “In vivo” is defined as biological processes, reactions or experiments that occur within the organism.

“Position” generally refers to a particular nucleotide by its index relative to a contextual zero-position, e.g., the first nucleic acid at the 5′ end of the molecule.

“Region” generally refers to a portion of a nucleic acid, wherein said portion is smaller than or equal to the entire nucleic acid.

The “secondary structure” of a nucleic acid (e.g., an RNA molecule) generally refers to the pattern of predicted or observed base pairing in the molecule. Base pairs may comprise canonical (Watson-Crick), Hoogsteen, wobble, sugar edge, or noncanonical base pairs. Base pairs may be between any edges (Watson-Crick, Hoogsteen, sugar, or C—H) of two nucleotides. Secondary structures may comprise one or more secondary structure motifs. Secondary structure motifs generally refer to well-stereotyped patterns of paired or unpaired nucleotides that recur across a plurality of RNA molecules. Non-limiting examples of secondary structure motifs include helices, hairpin loops or stem loops, internal loops or bulges, junction loops or multiway junctions, terminal mismatches, and single nucleotide overhangs. A secondary structure may be predicted by a computational algorithm. Non-limiting examples of packages implementing such algorithms include ViennaRNA, NUPACK, RNAstructure, RNAsoft, CONTRAfold, CycleFold, LearnToFold, MXfold, and SPOT-RNA. A secondary structure may be obtained from atomic (3D) coordinates of an RNA molecule. A secondary structure may be presented by, for example, a base-pairing matrix or a “dot-bracket” string.

“Tertiary structure” and “3D structure” of a nucleic acid (e.g., an RNA molecule) are used interchangeably herein to generally refer to the predicted or observed atomic coordinates of the nucleic acid. Certain elements or regions of an RNA molecule may be referred to herein as “tertiary motifs.” Tertiary motifs generally refer to well-stereotyped 3D motifs that recur across a plurality of RNA molecules. Non-limiting examples of tertiary motifs include pseudoknots, multi-way junctions, coaxial stacks, a-minor motifs, kissing stem-loops, ribose zippers, and tetraloop/tetraloop receptors. Tertiary structures may be determined from experimental methods such as, for example, X-ray crystallography, electron microscopy, and nuclear magnetic resonance spectroscopy. Tertiary structures may be predicted from sequence and other data or representations of an RNA molecule, such as by methods as systems disclosed herein.

The term “sequence identity” or “percent identity” in the context of two or more nucleic acid or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for nucleic acid sequences include, e.g., BLASTN using parameters of a word-size (W) of 28, an expectation threshold (E) of 0.05, and a reward/penalty ratio of 1/−2 (these are the default parameters for BLASTN in the BLAST suite available at https://blast.ncbi.nlm.nih.gov).

As used herein, “chemical mapping” generally refers to one or more techniques used to probe the solvent accessibility or conformational flexibility, interactions between pairs of nucleotides in a sequence, or any other mechanism that results in a probing signal that depends, in part, on 3D structure, of at least a part of a molecule (e.g., a nucleic acid, such as an RNA molecule or part thereof). Chemical mapping may comprise contacting a molecule with a chemical probing agent and measuring a signal indicative of a reaction with the chemical probing agent at a position or region (e.g., nucleotide or atom) of the molecule. Non-limiting examples of chemical probing agents include methylating agents, such as dimethyl sulfate (DMS), and acylating agents. In some cases, an acylating agent comprises a selective 2′-hydroxyl acylation and primer extension (SHAPE) reagent. In some cases, the SHAPE reagent comprises 1-methyl-7-nitroisatoic anhydride (1M7), 1-methyl-6-nitroisatoic anhydride (1M6), 5-nitroisatoic anhydride (5NIA), or N-methyl-nitroisatoic anhydride (NMIA). In some embodiments, the chemical probing agent comprises 2A3. In some cases, measuring the signal may comprise using a reverse-transcriptase that has increased propensity for mismatches and/or terminations at modified locations, and then sequencing the resulting DNA to estimate the chemical mapping signal.

### Overview

Chemical mapping experiments have been used to complement experimental structural biology approaches for RNA secondary structure prediction. Information on secondary structure can be used to improve the accuracy of tertiary structure prediction. In chemical mapping experiments, RNA molecules are exposed to chemicals that induce small changes in the RNA molecules. Following further experimental treatment, these changes can be measured, for example through DNA sequencing, an approach that is relatively inexpensive and amenable to high-throughput experiments. However, the resulting data is noisy, and the detailed processes that lead from the 3D structure and its exposure to a chemical agent to the experimental data have resisted a predictive understanding. Instead, these data are used to guide secondary structure prediction using heuristics based on the observation that Watson-Crick base-paired bases are less sensitive to chemical mapping than unpaired ones.

In parallel, computational methods for RNA tertiary structure prediction from nucleotide sequence have been developed. These include template-based modeling approaches, approaches that build on sampling of candidate structures and subsequent selection among these candidate structures, and recently, deep-learning-based approaches. Although these approaches may also make use secondary-structure inferred from chemical mapping data, they do not integrate chemical mapping data to improve the accuracy of tertiary structure prediction, for example if the RNA for which chemical mapping data is available and the RNA for which one desires to predict a tertiary structure are of substantially different sequence. Moreover, methods/algorithms for tertiary structure prediction have not been described in the art, that teach ingesting of chemical mapping data and using it to directly improve tertiary structure prediction. This may be due to lack of available chemical mapping data at scale, and lack of utility/efforts in collecting and using such data traditionally. Thus, it is not routine to use chemical mapping data to directly predict RNA tertiary structures (e.g., without relying on an intermediate secondary structure prediction) and the use of machine-learning techniques to allow such was not previously possible.

Described herein are methods and systems for predicting the tertiary structure of nucleic acids (e.g., RNA). Methods and systems may comprise one or more machine learning algorithms trained on data characterizing one or more reference RNA molecules. The trained machine learning algorithm may then be applied to an RNA molecule of interest to predict a tertiary structure of the RNA molecule of interest.

A machine learning algorithm may be trained at least in part on data comprising chemical mapping data. The chemical mapping data may comprise data indicative of which parts (e.g., nucleotides or atoms) of a nucleic acid (e.g., RNA) molecule are protected from attack by a chemical modifier (e.g., chemical probing agent). In some embodiments, the chemical modifier comprises dimethyl sulfate (DMS). In some embodiments, the chemical modifier comprises a selective 2′-hydroxyl acylation and primer extension (SHAPE) reagent. In some embodiments, the SHAPE reagent comprises an acylating agent such as 1-methyl-7-nitroisatoic anhydride (1M7), 1-methyl-6-nitroisatoic anhydride (1M6), 5-nitroisatoic anhydride (5NIA), or N-methyl-nitroisatoic anhydride (NMIA). In some embodiments, the chemical probing agent comprises 2A3.

Methods and system of the present disclosure may comprise one or more machine learning algorithms. The one or more machine learning algorithms may comprise one or more artificial neural networks (ANNs). ANNs with different architectures may be combined to process or predict data of one or more modalities indicative of a nucleic acid's tertiary structure. For example, a recurrent neural network (RNN), transformer, or other attention network architecture may be used to process sequence data while a graph neural network may be used to predict or refine 3D structures. In particular, ANNs of the present disclosure may comprise layers which are equivariant to rigid body rotations and translations in 3D, making them particularly suitable for the learning and prediction of molecular structures.

Compared to other methods of predicting RNA structures, methods of the present disclosure may provide certain benefits and advantages. Methods of the present disclosure may be configured to predict tertiary structures directly from chemical mapping data (e.g., without needing to compute or accept as input a secondary structure of the target molecule). Determining a secondary structure from chemical mapping data (or require such a determination to predict tertiary structure) may erroneously predict a nucleotide to be base-paired when it is actually participating in a high-order (e.g., tertiary) structural motif or is unpaired but its chemical mapping signal is affected by other nearby atoms. These errors may occur because residues which give a high chemical mapping signal are generally presumed in other methods to be base-paired. However, any region of an RNA molecule which, for example, has reduced solvent accessibility or conformational flexibility, such as by virtue of participating in a tertiary structural motif interaction, may show a relatively low chemical mapping signal, even though the region is not base-paired. Additionally, methods of the present disclosure may be configured to predict tertiary structures of RNA molecules for which no chemical mapping data is available, even those with low sequence identity compared to the molecules used to train the machine learning algorithm(s). In some embodiments, the method can comprise predicting the predicted tertiary structure of the RNA molecule of interest based on the chemical mapping data for the RNA molecule of interest. In some embodiments, the method can comprise predicting the chemical mapping data for the RNA of interest and the predicted tertiary structure of the RNA of interest using the same embedding (e.g., as output from another machine learning algorithm that processes an RNA sequence).

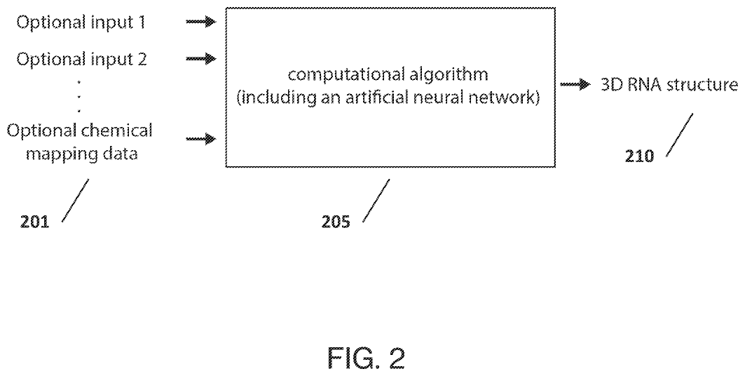

An example system 200 is depicted in FIG. 2. System 200 is configured to process one or more inputs 201 of the same or different modalities. The inputs 201 may comprise one or more RNA sequences whose structures are to be predicted by system 200. The inputs 201 may additionally comprise other information indicative of an RNA molecule's structure. For example, inputs 201 may comprise one or more of chemical mapping data for one or more of the input RNA sequences, sequence alignment or conservation information for one or more of the input RNA sequences, secondary structure information for one or more of the input RNA sequences, or one or more candidate or reference (e.g., experimentally determined) tertiary structures.

In some embodiments, the training set may comprise sequences with data characterizing their secondary structures. This data may be used in various forms, such as in the form of base-pair-probability matrices, either as pairwise input features to the algorithm or as an additional prediction target (e.g., analogously to the case of the 2D chemical mapping data described elsewhere herein). In some embodiments, the secondary structure data may or may not be used to optimize the structure module during training (e.g., to improve tertiary structure prediction capabilities), as described herein below. Various secondary structures may be characterized in the training set, e.g., annotations of stems, hairpin loops, pseudoknots, bulges, internal loops, multiloops, etc., for one or more elements in the sequences.

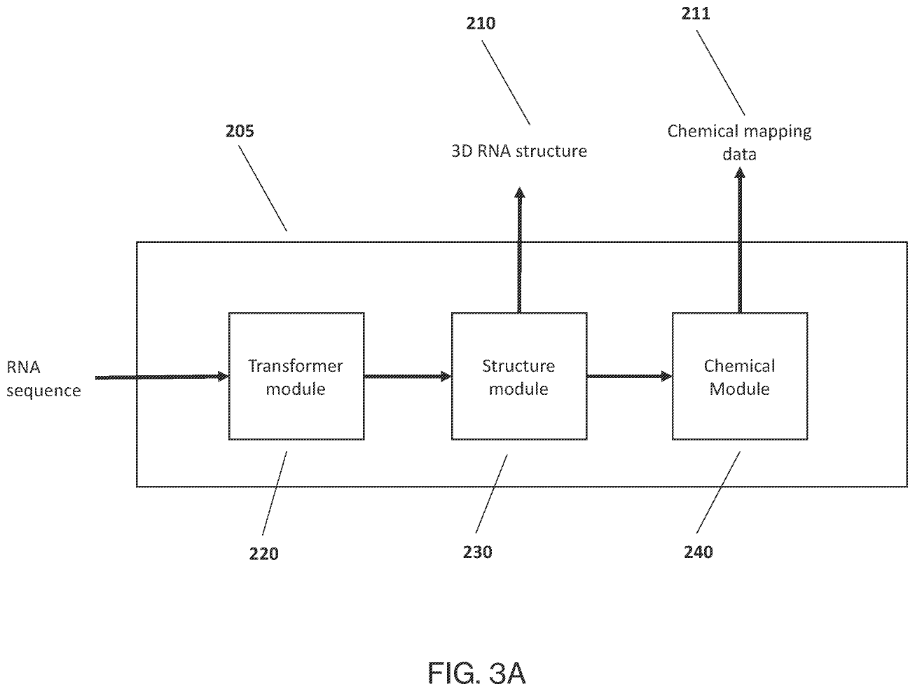

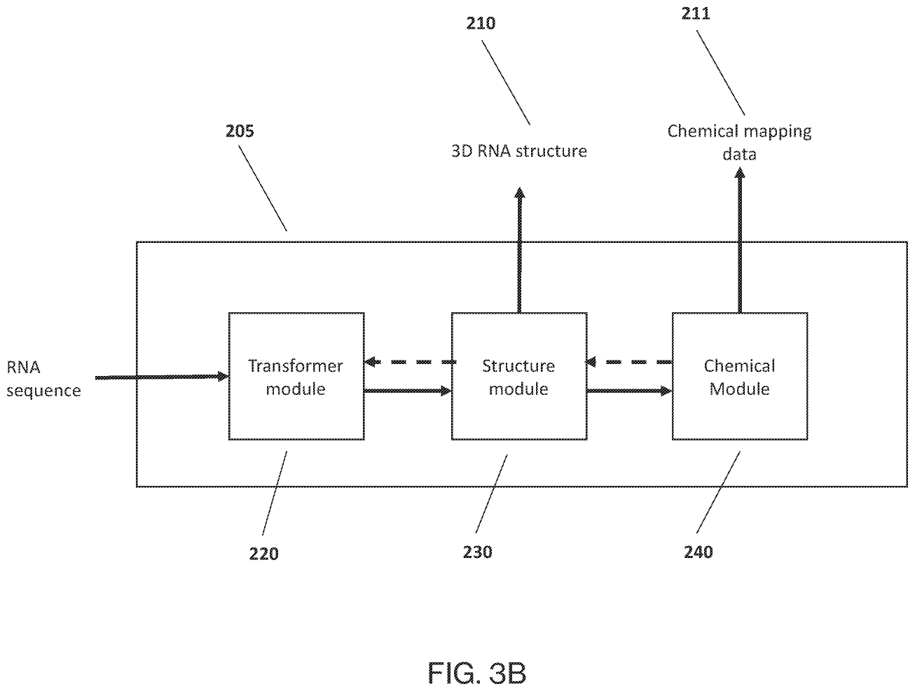

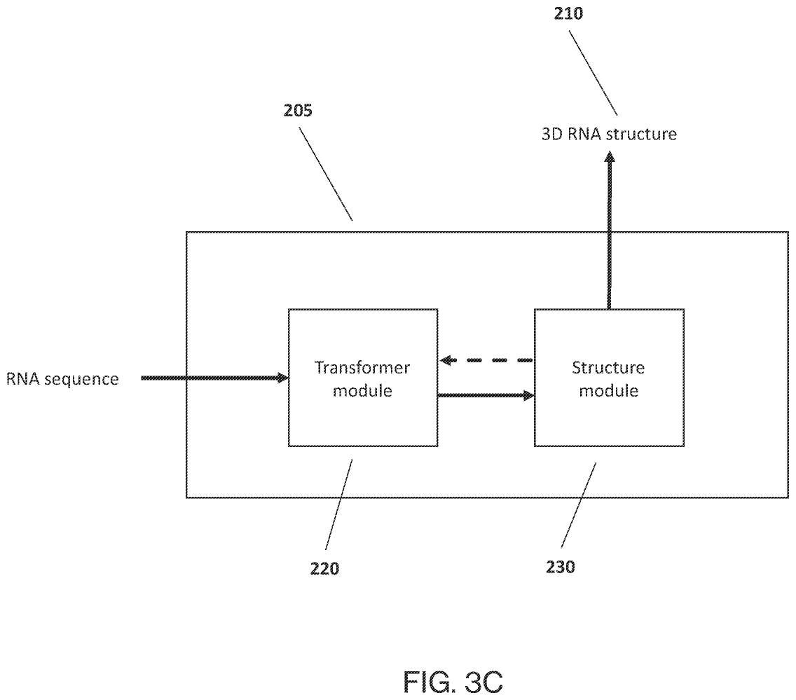

The inputs 201 are processed by computational algorithm 205 to generate a predicted 3D structure 210 corresponding to the inputs. The computational algorithm 205 may comprise one or more artificial neural networks (ANNs) trained on a plurality of RNA sequences and tertiary structures. The computational algorithm 205 may comprise multiple machine learning algorithms (or modules) configured to process data of different modalities. Additionally, the input of one machine learning algorithm may comprise the outputs of one or more other machine learning algorithms and the output of one machine learning algorithm may compose the input to one or more other machine learning algorithms. The computational algorithm may perform additional predictions on the RNA sequences and produce corresponding outputs. In an example illustrated in FIG. 3A, the computational algorithm 205 comprises a transformer module 220, a structure module 230, and a chemical module 240. The transformer module 220 is configured to receive the input RNA sequence. The transformer model 220 may receive an encoding (e.g., a one-hot encoding) or embedding of the RNA sequence. The system may comprise one or more additional modules (e.g., a deep neural network) configured to generate the encoding or embedding (not pictured).

The transformer module 220 may comprise an attention network comprising an attention mechanism (e.g., one or more attention layers) configured to process the RNA sequence (or representation thereof, such as an embedding). The transformer 220 may update the sequence embedding as well as a second, pairwise embedding of residues which encodes information about the relationship between or proximity of pairs of residues. The transformer 220 may be configured to update the pairwise embedding based at least in part on the sequence embedding. The transformer 220 may output the updated sequence embedding and pairwise embedding. The output updated sequence embedding and pairwise embedding may be used by other machine learning algorithms composing computational algorithm 205.

In an example, output sequence embedding and pairwise embedding from the transformer 220 may then be input to a structure module 230, as further illustrated in FIG. 3A. The structure module 230 may comprise an attention mechanism. The structure module 230 may comprise machine learning algorithms (e.g., ANNs) that employ geometry-aware and equivariant attention operations, as described elsewhere herein, to generate a representation of the 3D structure of the input RNA. The structure module 230 may predict the 3D coordinates of the RNA molecule from the sequence embedding and the pairwise embedding. In an example, the structure module 230 predicts rotations and translations for coordinates of at least some atoms (e.g., C4′, C1′, N1/N9) from a local residue frame to a global molecule frame (e.g., main frame) of at least a subset of residues of the RNA molecule to produce a representation of the predicted RNA tertiary structure. In some embodiments, the structure module 230 may predict coordinates of every atom in the target RNA. Alternatively, the structure module 230 may predict coordinates of only a subset of the atoms in the target RNA. The subset of atoms may comprise at least one atom from every nucleotide of the target RNA, or the subset may comprise atoms only from certain regions (e.g., nucleotides) of the target RNA. As at least part of its output, the computational algorithm 205 may output the predicted 3D structure 210 of the target sequences.

In some embodiments, the tertiary structure representation comprises a different (sub)set of atoms. The main frame representation may comprise different atoms and not be identical for each nucleotide in the RNA molecule. It may range from zero atoms to a complete list of atoms at the level of individual nucleotides. The structure representation may be without an explicit embedding in terms of 3D coordinates at the level of distance maps that are represented as part of the pairwise features in the structure module. This distance map representation may be augmented with angles, dihedrals, or both.

In an example, computational algorithm 205 may further comprise a chemical module 240, as illustrated in FIG. 3A. The chemical module may be configured to predict chemical mapping data 211 for the input RNA molecule(s) based at least in part on the predicted 3D structure (or a representation or embedding thereof). In an example, chemical module 240 comprises a message-passing graph neural network (MPGNN) that accepts the predicted RNA structure as input. In another example, the MPGNN for the chemical module may be replaced with a transformer. The chemical module 240 may operate on a graph representation of the predicted RNA structure in which at least a subset of the atoms (e.g., C4′, C1′, and N1/N9) are represented by the graph nodes and the edges of the graph are drawn between atoms close in Euclidean space (e.g., within a certain cutoff, such as 15 Å). The chemical model 240 may update the graph through a series of message passing steps to generate the predicted chemical mapping data 211. Additionally, or alternatively, chemical module 240 may comprise an equivariant neural network architecture such as point-convolution architectures or equivariant message-passing architectures, as described elsewhere herein.

FIG. 3B depicts information flow during training of the example system illustrated in FIG. 3A. Training the system 205 may comprise providing a training data set comprising one or more reference RNA molecules with known chemical mapping data. The training data (or a subset thereof) can be fed through the (untrained) system in the forward direction (indicated by solid arrows) to generate predicted outputs. The discrepancy between the predicted output and the known output may be quantified by a loss function. The choice of loss function may be based in part on the type of output data. In the example depicted in FIG. 3B, an L2 function can be used to quantify the error between the predicted and observed chemical mapping data for a reference RNA molecule. Based on the quantified error, a gradient with respect to one or more parameters (e.g., weights, biases, threshold values) of the computational algorithm (e.g., machine learning algorithm) may be computed by, for example, backpropagation to update the parameters of the neural network (indicated by dashed arrows) so that the output (predicted) values are consistent with the known values.

In another example, depicted in FIG. 3C, the system is trained by quantifying errors in the predicted tertiary structures of the RNA molecules in the training data set (or subset thereof). In this example, the loss function may comprise, for example, (root) mean-square deviation between predicted and superposed reference structure, global distance test (GDT) score, residue-residue contact area distance (CAD) score, or Local Difference Distance Test (LDDT) score, or another score well-suited to assessing structural similarity of biomolecular structures. The loss function may comprise a regression loss function. The loss function may comprise a logistic loss function. The loss function may comprise a variational loss. The loss function may comprise a prior. The loss function may comprise a Gaussian prior. The loss function may comprise a non-Gaussian prior. The loss function may comprise an adversarial loss. The loss function may comprise a reconstruction loss. The loss function may quantify differences, between a predicted and superimposed reference structure, in pairwise distances between nucleotides i and j (e.g., via one or more atoms of the nucleotides, center-of-mass of the nucleotide, etc.) for each pair of nucleotides. The loss function may quantify differences, between a predicted and superposed reference structure, in angles formed by a sequence of three nucleotides, ijk. The loss function may quantify differences, between a predicted and superposed reference structure, in dihedrals (torsional angles) formed by a sequence of four nucleotides, ijkl. The loss function may quantify differences, between a predicted and superposed reference structure, in improper dihedrals formed by four nucleotides where three nucleotides are bonded to a central nucleotide, ijkl.

Aside from loss function, training may also comprise choosing, for example, a set of initial parameter values (or a method of generating them) and an optimization scheme. In some embodiments, the initialization comprises Xavier initialization. In some embodiments, the optimization comprises Adam optimization.

The methods and systems described herein may operate on any nucleic acid sequence. In some embodiments, the target nucleic acid sequences are RNA sequences. In some embodiments, the target RNAs are coding RNAs. In some embodiments, the target RNAs are non-coding RNAs (ncRNAs). In some embodiments, the target RNAs are circular RNAs. In some embodiments, the target RNAs are transfer RNAs (tRNAs), ribosomal RNAs (rRNAs), messenger RNAs (mRNAs), small nuclear RNAs (snRNAs), small nucleolar RNAs (snRNAs), micro RNAs (miRNAs), short-hairpin RNAs (shRNAs), small interfering RNAs (siRNAs), Y RNAs, vault RNAs, antisense RNAs, transcription initiation RNAs (tiRNAs), transcriptional start-site associated RNAs (TSSa-RNAs), piwi-interacting RNAs (piRNAs), guide RNAs (gRNAs), or ribozymes. The target RNAs may comprise single stranded RNAs (ssRNAs) or double stranded RNAs (dsRNAs). In some embodiments, the target RNAs comprise a transcriptome or part thereof. In some embodiments, the transcriptome comprises a transcriptome from a eukaryote, prokaryote, or archaea. In some embodiments, the transcriptome comprises a transcriptome from a fungus, bacterium, virus, protist, alga, plant, or animal. In some embodiments, the transcriptome comprises a human transcriptome. In some embodiments, the target RNAs comprise RNAs that are synthetic or not normally present in nature. In some embodiments, the target RNAs comprise natural RNA sequences. In some embodiments, the machine-learning model can be fine-tuned to generate a more accurate tertiary structure for a RNA molecule of interest. After the machine-learning model has been trained on an initial dataset, chemical mapping data of the RNA molecule of interest can be used to fine-tune the machine learning algorithm. By updating parameters of the machine learning based on the chemical mapping data of the RNA molecule, the model can output a more accurate tertiary structure of the RNA molecule of interest. In some embodiments, the machine-learning model can be fined tuned using chemical mapping data from 1, 2, 3, 4, 5, 6, 7, 8, 9, 10 or any number of RNA molecules of interest. The RNA molecules of interest may be related sequences comprising a relatively high sequence similarity, therefore, the inclusion of more RNA molecules of interest may further improve the accuracy of the machine learning algorithm.

In some aspects, the present disclosure provides a method for determining a target region or subsequence in a RNA molecule of interest. The target region or subsequence can be a portion of the RNA molecule of interest where, if a pharmaceutical drug binds thereto, can perturb the structure of the RNA molecule of interest and enhance or disrupt its biological function. The target can be identified based on the predicted tertiary structure. In some embodiments, the pharmaceutical drug that binds to the RNA molecule of interest can be formulated based on the predicted tertiary structure.

### Chemical Mapping Data

Methods and systems as described herein may comprise, ingest, operate on, or output one or more datasets comprising chemical mapping data. Chemical mapping datasets may comprise data indicative of the reactivity of a region (e.g., nucleotide or atom) of a molecule (e.g., RNA) toward a chemical probing agent. Chemical mapping datasets may comprise indications of conformational flexibility or solvent accessibility of one or more nucleotides or subsequences of a sequence. Chemical mapping datasets may comprise indications of interactions between pairs of nucleotides in a sequence. Chemical mapping datasets may comprise molecular binding data (biomolecules as well as more generally organic and inorganic molecules. In some embodiments, the chemical probing agent comprises a methylating agent, such as dimethyl sulfate. In some embodiments, the chemical probing agent comprises an acylating agent, such as a selective 2′-hydroxyl acylation and primer extension (SHAPE) reagent. In some embodiments, the chemical mapping data comprise experimentally observed reactivities. In some embodiments, the chemical mapping data comprise predicted reactivities

In some embodiments, chemical mapping data comprise dimethyl sulfate (DMS) reactivity data. DMS can methylate unpaired cytosine and adenine residues in an RNA molecule but has reduced reactivity toward base-paired (or otherwise less solvent accessible or conformationally flexible) cytosine and adenine residues. A DMS reactivity profile (e.g., characterizing reactivity of one or more residues or other regions toward DMS) of an RNA molecule can be obtained by treating the RNA molecule with DMS, reverse transcribing the methylated RNA with reverse transcriptase, and sequencing the resulting DNA. Reverse transcriptase frequently incorporates an incorrect DNA nucleotide, inserts additional nucleotide(s), deletes nucleotide(s) and/or aborts termination when it encounters a methylated RNA residue. Thus, the relative observed frequency of these mutations at a given position is generally correlated with the conformational flexibility or solvent accessibility of a given nucleotide. DMS-reactivity profiles may also be determined by direct RNA sequence using, for example, a nanopore device.

In some embodiments, chemical mapping data comprise selective 2′-hydroxyl acylation and primer extension (SHAPE) reactivity data. A SHAPE reagent is generally an acylating agent which can acylate the 2′-hydroxyl of an unpaired nucleotide but has reduced reactivity toward a base-paired (or otherwise less solvent accessible of conformationally flexible) nucleotide. A SHAPE reactivity profile (e.g., characterizing reactivity one or more residues or other regions to a SHAPE reagent) of an RNA molecule can be obtained by treating the RNA molecule with a SHAPE reagent and reverse transcribing the modified RNA to generate cDNA. Reverse transcriptase frequently incorporates an incorrect DNA nucleotide, inserts additional nucleotide(s), deletes nucleotide(s) and/or aborts termination when it encounters an acylated RNA residue. Quantification of the lengths or mutations of the cDNAs in the cDNA pool thus allows for a readout of which regions (e.g., nucleotides) of the RNA molecule show the most conformational flexibility or solvent accessibility. SHAPE-reactivity profiles may also be determined by direct RNA sequence using, for example, a nanopore device.

In some embodiments, chemical mapping data comprise reactivity data of an RNA molecule and one or more RNA molecules derived from the RNA molecule toward one or more chemical probing agents. The derived RNA molecules may comprise point mutations relative to the RNA molecule. Such mutations may be generated randomly, such as through the use of error-prone polymerases, or the derivative RNAs may be rationally designed with certain point mutations. An example method that generates such data is mutate-and-map readout through next generation sequencing (M2-seq). M2-seq and other multidimensional chemical mapping experiments can indicate which nucleotides respond to perturbations (e.g., chemical modification by a chemical probing agent) at every other nucleotide, allowing inference of which pairs of nucleotides interact in an RNA structure. Machine learning algorithms (e.g., ANNs) as disclosed herein may be configured to ingest, operate on, output, or predict multidimensional chemical mapping data.

The chemical mapping data may comprise chemical reactivity data characterizing reactivity of regions of an RNA molecule(s) toward one chemical probing agent. Alternatively, the chemical mapping data may comprise chemical reactivity data characterizing reactivity of regions of an RNA molecule(s) toward more than one chemical probing agent. In an example, the chemical mapping data comprise DMS reactivity data for a plurality of RNA molecules. In another example, the chemical mapping data comprise SHAPE reactivity data for a plurality of RNA molecules. In yet another example, the chemical mapping data comprise DMS and SHAPE reactivity data for a plurality of RNA molecules.

Data for training a machine learning algorithm as described herein may comprise chemical mapping data for one or more reference molecules (e.g., RNA molecules). In some embodiments, the training data comprises chemical mapping data for at least about 10, 50, 100, 200, 300, 400, 500, 600, 700, 800, 900, 1,000, 2,000, 5,000, 10,000, 100,000, 1,000,000, 10,000,000, or more reference molecules. In some embodiments, the training data comprises chemical mapping data for no more than about 1,000,000, 10,000,000, 100,000, 10,000, 5,000, 2,000, 1,000, 900, 800, 700, 600, 500, 400, 300, 200, 100, 50, 10, or fewer reference molecules. In some embodiments, the chemical mapping data comprise chemical mapping data for a target RNA molecule. In some embodiments, the chemical mapping data do not comprise chemical mapping data for the target molecule. In some embodiments, the chemical mapping data comprise multidimensional chemical mapping data.

In some embodiments, the one or more reference RNA molecules are not related to the target RNA molecule. The reference RNA molecules may comprise no more than about 80%, 70%, 60%, 50%, 40%, 30%, 20%, or less identity to the target RNA molecule. Alternatively, the one or more reference RNA molecules may be related to the target molecule. The reference RNA molecules may comprise at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more identity to the target RNA molecule.

In some embodiments, the chemical mapping data are collected from in vitro experiments. In some embodiments, the chemical mapping data are collected from in vivo experiments. In some embodiments, the one or more reference RNA molecules are synthetic RNAs. In some embodiments, the one or more reference RNA molecules are natural RNAs. In some embodiments, the one or more reference RNA molecules comprises a mixture of synthetic RNAs and natural RNAs.

### Tertiary Structure Prediction

Methods and system of the present disclosure may comprise one or more machine learning algorithms. The one or more machine learning algorithms may comprise one or more artificial neural networks (ANNs). ANNs with different architectures may be combined to process or predict data of one or more modalities indicative of a nucleic acid's tertiary structure. For example, a recurrent neural network (RNN), transformer, or other attention network architecture may be used to process sequence data while a graph neural network may be used to predict or refine 3D structures. In particular, ANNs of the present disclosure may comprise layers which are equivariant to rigid body rotations and translations in 3D, making them particularly suitable for the learning and prediction of molecular structures. Methods of the present disclosure may be configured to predict tertiary structures directly from chemical mapping data (e.g., without needing to compute or accept as input a secondary structure of the target molecule).

The structure model of a machine learning algorithm can output a predicted tertiary structure. The tertiary structure can be refined iteratively based on multiple passes through the structure module. For example, the output from a structure module can be recursively fed as input to the structure model to output an updated tertiary structure. The machine learning algorithm can be configured to receive a tertiary structure and predict a set of one or more updates which can be added to the tertiary structure. The updates can be updates to 3D coordinates, angles, dihedrals, a distance histogram, or any other measure of 3D structure.

The iterative process can end after meeting a convergence criteria. The convergence criteria can be, e.g., when the updated tertiary structure is substantially the same as the previous tertiary structure. Such a criteria can be based on the magnitude of the difference between the updated tertiary structure and the previous tertiary structure. For example, if using pairwise distances in a convergence criteria, the convergence criteria may be formulated as when the update is less than 0.1 Angstroms per pair on average (various other threshold values may be used). Likewise, a convergence criteria may be formulated for angles, dihedrals, 3D coordinates, or any other measure of 3D structure. In some embodiments, the machine learning algorithm may be configured to output a measure of its accuracy or confidence. For example, a machine learning algorithm can be designed such that it outputs an indication of accuracy or confidence based on the examples it has seen in the training dataset. In some embodiments, the machine learning algorithm can output the local accuracy of the structure prediction (which can be measured based on, e.g., the metric of predicted Local Distance Difference Test (pLDDT)). The local accuracy can be used as a convergence criteria, where the algorithm updates the tertiary structure until a threshold value for local accuracy is met, or until it no longer substantially improves. The algorithm's ability to itself predict the quality of its tertiary structure predictions can be trained based on the ground-truth tertiary structure data provided during training and the LDDT metric that can be computed in comparison of tertiary structure data and tertiary structure prediction.

### Machine Learning Algorithms

Methods and systems as described herein may comprise one or more machine learning algorithms. The machine learning algorithm may comprise an unsupervised machine learning algorithm. The trained algorithm may comprise a supervised machine learning algorithm. The machine learning algorithm may comprise a self-supervised machine learning algorithm.

In some embodiments, a machine learning algorithm of methods and systems as described herein utilizes one or more artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network (DNN)) is an ANN comprising a plurality of hidden layers, e.g., two or more hidden layers. Each layer of the neural network may comprise a number of nodes (or “neurons”). A node receives input that comes either directly from the input data or the output of nodes in previous layers, and performs a specific operation (e.g., a summation operation). A connection from an input to a node is associated with a weight (or weighting factor). The node may sum up the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

Non-limiting examples of structural components of machine learning algorithms described herein include convolutional neural networks (CNNs), deep neural networks (DNNs), recurrent neural networks (RNNs), long short-term memory networks (LSTMs), attention networks, and transformers, graph neural networks (GNNs), message passing neural networks (MPNNs), and combinations or variations thereof.

In some embodiments, a neural network comprises a series of layers, wherein each layer comprises individual units termed “neurons.” In some embodiments, a neural network comprises an input layer, to which data is presented; one or more internal, or “hidden,” layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined, for example, by the problem complexity, and the maximum number may be limited, for example, by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments, a machine learning algorithm comprises a CNN. The CNN may be a deep or feedforward ANNs. The CNN may be applicable to analyzing sequence data. The CNN may comprise an input, an output layer, and multiple hidden layers. The hidden layers of a CNN may comprise convolutional layers, pooling layers, fully-connected layers and normalization layers.

The convolutional layers may apply a convolution operation to the input and pass results of the convolution operation to the next layer. The convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels) comprising one or more learnable weights. The learnable filters may have a small receptive field and extend through the full depth of the input volume. During the forward pass, each filter may be convolved across the width and height of the input volume, compute the dot product between the entries of the filter and the input, and produce a two-dimensional activation map of that filter. As a result, the network may learn filters that activate when it detects some specific type of feature at some spatial position in the input.

In some embodiments, a machine learning algorithm comprises an RNN. RNNs are neural networks with cyclical connections that can encode and process sequential data, such as a sequence of an RNA molecule. An RNN can include an input layer that is configured to receive a sequence of inputs. An RNN may additionally include one or more hidden recurrent layers that maintain a state. At each step, each hidden recurrent layer can compute an output and a next state for the layer. The next state may depend on the previous state and the current input. The state may be maintained across steps and may capture dependencies in the input sequence.

An RNN can be a long short-term memory (LSTM) network. An LSTM network may be made of LSTM units. An LSTM unit may include of a cell, an input gate, an output gate, and a forget gate. The cell may be responsible for keeping track of the dependencies between the elements in the input sequence. The input gate can control the extent to which a new value flows into the cell, the forget gate can control the extent to which a value remains in the cell, and the output gate can control the extent to which the value in the cell is used to compute the output activation of the LSTM unit.

Alternatively, a machine learning algorithm can comprise a transformer. A transformer may be a model without recurrent connections. Instead, it may rely on an attention mechanism. Attention mechanisms may focus on, or “attend to,” certain input regions while ignoring others. This may increase model performance because certain input regions may be less relevant. At each step, an attention unit can compute a dot product of a context vector and the input at the step, among other operations. The output of the attention unit may define where the most relevant information in the input sequence is located.

In some embodiments, a machine learning algorithm comprises a graph neural network (GNN). GNN are neural networks which are specifically designed to perform inference on graph-based data. GNNs as employed in methods and systems herein may be graph convolution networks (GCNs), graph attention networks (GATs), message passing neural networks, or other GNNs that perform permutation-invariant pooling and aggregation.

In some embodiments, ANNs as described herein may comprise one or more equivariant neural networks, such as point-convolution architectures or equivariant graph neural networks. These and related architectures may comprise neural network layers which are equivariant to translations and rotations and are thus well-suited to learning from or predicting molecular structural (e.g., RNA tertiary structure) data.

The weighting factors, bias values, and threshold values, or other computational parameters of the neural network, may be “taught” or “learned” in a training phase using one or more sets of training data. Training a neural network can involve providing inputs to the untrained neural network to generate predicted outputs, comparing the predicted outputs to the expected outputs, and updating the neural network's parameters to account for the difference between the predicted outputs and the expected outputs. A loss function can be used to quantify a difference between the predicted outputs and the expected outputs. Based on the calculated difference, a gradient with respect to each parameter may be calculated by backpropagation to update the parameters of the neural network so that the output value(s) that the ANN(s) computes are consistent with the examples included in the training dataset. This process may be iterated for a certain number of iterations or until some stopping criterion is met.

The choice of loss function for a particular neural network may be based in part on the type of data the neural network is configured to process. For example, a neural network (such as a MPGNN) configured to predict chemical mapping data for an input tertiary RNA structure may be trained to optimize an L2 (squared error) loss. In another example, a neural network may be trained to optimize an L1 (absolute error) or cross-entropy loss. In yet another example, a neural network may be trained to optimize a function or score that quantifies structural similarity between two or more molecules. The loss function may comprise, (root) mean-square deviation between predicted and superposed reference structure, global distance test (GDT) score, residue-residue contact area distance (CAD) score, or Local Difference Distance Test (LDDT) score.

Systems and methods as described herein may use more than one machine learning algorithm to determine an output (e.g., chemical mapping data or tertiary structure of an RNA molecule). Systems and methods may comprise 1, 2, 3, 4, 5, 6, 7, 8, 9, 10, 11, 12, 13, 14, 15, 16, 17, 18, 19, 20, or more machine learning algorithms. A machine learning algorithm of the one or more machine learning algorithms may be trained on a particular type of data (e.g., chemical mapping data, alignment or conservation data, tertiary structure data). Alternatively, a machine learning algorithm may be trained on more than one type of data. The inputs of one machine learning algorithm may comprise the outputs of one or more other machine learning algorithms. Additionally, a machine learning algorithm may receive as its input the output of one or more machine learning algorithms.

### Computing System

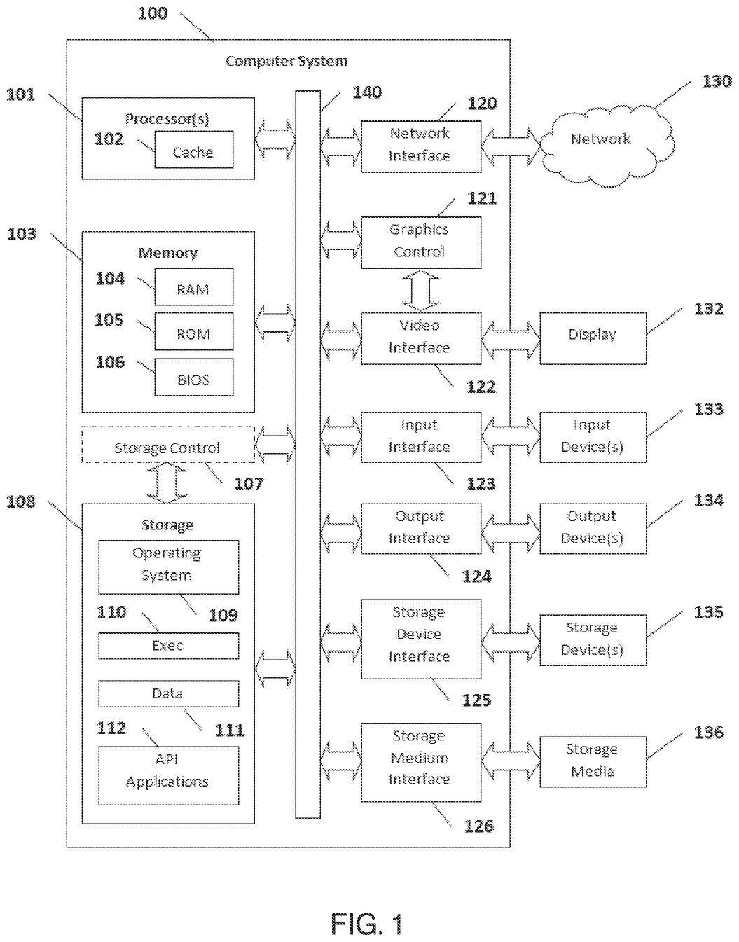

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. A computing system can be configured to receive or transmit requests for predicting tertiary structure of an RNA molecule of interest, and/or receive or transmit a predicted tertiary structure an RNA molecule of interest. In some embodiments, the computing system can implement a method comprising sending a query for predicting the tertiary structure of the RNA molecule of interest to a computer comprising a machine-learning model. The machine learning algorithm can be configured to generate the tertiary structure. The machine-learning model could have been trained by a process including (i) creating a training data set comprising chemical mapping data for a first plurality of RNA molecules and tertiary structure data for a second plurality of RNA molecules, (ii) training the machine-learning model using the training data set, or both. The computer-implemented method can comprise receiving the predicted tertiary structure of the RNA molecule of interest from the computer. In some embodiments, the computing system can implement a method comprising receiving a query for predicting the tertiary structure of the RNA molecule of interest using a machine-learning model. The machine learning algorithm can be configured to generate the tertiary structure. The machine-learning model could have been trained by a process including (i) creating a training data set comprising chemical mapping data for a first plurality of RNA molecules and tertiary structure data for a second plurality of RNA molecules, (ii) training the machine-learning model using the training data set, or both. The computer-implemented method can comprise generating the predicted tertiary structure of the RNA molecule of interest from the computer. The computer-implemented method can comprise transmitting the predicted tertiary structure of the RNA molecule of interest from the computer.

The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, distributed and cloud computing platforms, server clusters, server computers, desktop computers, laptop computers, notebook computers, sub-notebook computers, and netbook computers.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of nucleic acid (e.g., RNA) structure, sequence, and chemical mapping information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### EXAMPLES

### Example 1—System for Predicting RNA Tertiary Structure from Chemical Mapping Data

A training set is created based on i) 500 RNA nucleotide sequences and corresponding experimentally determined tertiary structures obtained from the Protein Data Bank (PDB) and ii) 10,000 RNA sequences for which 1D chemical mapping data in the form of site-specific reactivities through dimethyl sulfate (DMS) exposure of the corresponding RNA molecules, reverse transcription to DNA, and DNA sequencing is available.

The training set is used to train a machine learning algorithm for RNA tertiary structure prediction for which the information flow is depicted in FIGS. 3A and 3B. The numerical representation of a given RNA nucleotide sequence is processed by two subparts of the algorithm referred to as the transformer module and the structure module, respectively. The output of the combined modules is a predicted RNA tertiary structure. This output is in turn the input to another part of the algorithm referred to as the chemical module. The output of this module is predicted chemical mapping data.

In this embodiment of the method and systems described herein, the neural network architecture comprises transformer and structure modules. The nucleotide sequence is encoded using a one-hot vector representation that is passed through a dense neural network layer to produce a numerical embedding. This embedding is the input to the transformer module that comprises a series of gated attention layers that iteratively update the sequence embedding and a second pairwise embedding at the level of nucleotides. The output sequence and pair representations are the input to the structure module that predicts rotation and translation matrices for main frames defined by atoms of nucleotides (e.g., C4′, C1′, N1/N9), forming a representation of the RNA tertiary structure. The prediction of the rotation and translation matrices is based on a geometry-aware attention operation and invariant point attention. The application of the predicted rotation and translation matrices to the individual main frames yields the predicted tertiary structure.

The chemical module comprises a message-passing graph neural network that takes the predicted RNA tertiary structure as input. The atoms (C4′, C1′, N1/N9) represent the graph nodes and edges are drawn between each node and the nodes that are close in Euclidean space (e.g., within a certain cutoff, such as 15 Å) based on the tertiary structure. The Euclidean distances correspond to the edge features. Node features comprise the atom type (e.g., C4′, C1′, N1/N9) and the nucleotide type to which each atom corresponds encoded as one-hot. The node and edge features are updated through a sequence of message passing steps. The predicted chemical mapping data is output based on the value of the updated node and edge features, such as the average over all node features corresponding to a nucleotide in the case of the 1D chemical mapping data.

The neural network architecture going from RNA nucleotide sequence to RNA tertiary structure prediction to chemical mapping data is differentiable. As such, the network parameters of all modules may be optimized based on the training signal that is received by comparing the predicted chemical mapping data to the actual chemical mapping data for the part of the training set that comprises nucleotide sequences and chemical mapping data. Here, the Adam optimizer and the L2 loss are used for the chemical mapping data. This training of the algorithm is depicted in FIG. 3B. This first training procedures alternates with the training on the data for which we have nucleotide sequences and corresponding experimentally determined tertiary structures. This training of the algorithm is depicted in FIG. 3C. Here, the loss is calculated at the level of the tertiary structure (in terms of the LDDT metric) and the loss signal backpropagated through the transformer and structure modules to update their parameters.

The tertiary structure prediction algorithm can be trained using chemical mapping data without any tertiary structures for the RNA molecules in the chemical mapping dataset. In addition, after training, no chemical mapping data is necessary, and the now optimized algorithm can be used to make tertiary structure predictions for novel RNA sequences.

The parameters of the neural network architecture and the described training procedure are optimized using methods described elsewhere herein (e.g., withholding a subset of training data encompassing RNA sequences with known tertiary structure and evaluating the accuracy of the tertiary structure prediction algorithm for a given set of parameters based on this holdout data). These parameters can include the number and specification of the different neural network layers, the dimensionality of the learned embeddings, the choice of learning rates, the initialization of the learnable variables of the algorithm (such as Xavier initialization), the optimizer (such as Adam), the loss functions (such as L2 norm) and their relative weight in optimization, the use of dropout layers, and the number of message-passing steps and nearest-neighbors (e.g., for the chemical module which may comprise a graph neural network).

After training the algorithm, one or more RNA sequences of interest are provided to the algorithm to predict and output RNA tertiary structure and chemical mapping data.

### Example 2—Iterative Refinement of Predicted RNA Tertiary Structure

A system is trained and deployed as described in Example 1 to predict the tertiary structure of a target RNA molecule. The predicted tertiary structure is refined iteratively based on multiple passes through the structure module in which the pairwise nucleotide representation derived from the current tertiary structure prediction is provided as the new input to the structure module. This iterative process ends when the algorithm's own prediction on the local accuracy of the structure prediction (measured based on the metric of predicted Local Distance Difference Test (pLDDT)) converges. The algorithm's ability to itself predict the quality of its tertiary structure predictions is trained based on the ground-truth tertiary structure data provided during training and the LDDT metric that can be computed in comparison of tertiary structure data and tertiary structure prediction.

### Example 3—Multi-Task Setup for Prediction of RNA Tertiary Structure and Chemical Mapping Data

In some embodiments, chemical mapping data prediction need not be preceded by tertiary structure prediction but implemented as a multi-task prediction problem in which at least part of the machine learning algorithm is shared among the tasks. For example, a one-hot vector embedding of the sequence (e.g., of length 4 for each nucleotide) and a second pairwise representation can be used to start with, in which for each nucleotide pair the relative displacement between nucleotides (at the sequence level) is encoded as one-hot and clipped at a predetermined length (e.g. 65). Both the one-hot sequence representation as well as the pairwise representation can be further processed through linear layers to obtain initial embeddings, in the case of the pairwise representation after concatenating the displacement embedding for each nucleotide pair with the one-hot encodings of the nucleotide identities. These embeddings can be the input to a transformer module as described in Example 1. The sequence and pairwise outputs of this module can be then directed to task-specific heads, such as the described structure module for tertiary structure prediction and, as an example of chemical mapping data prediction, a linear neural network layer followed by a sigmoid nonlinearity that predicts a mutation probability for each position in the input sequence. The parameters of the transformer module can be optimized based on training data comprising tuples of both (sequence, chemical mapping) and (sequence, RNA tertiary structure) information, a procedure that can yield accuracy improvements for both tasks compared to training independent predictors for each task.

### Example 4—Sequential Training of Models for Chemical Mapping Data and RNA Tertiary Structure Prediction

A machine learning algorithm is first trained to predict chemical mapping data from RNA sequence using initial encodings and a transformer module similar to the one described in Example 3 but without the multi-task setup. For a given RNA sequence of interest, single and pairwise representations from the pretrained transformer module can then be extracted, processed by a linear layer to obtain the desired dimensionality, and these can be used as inputs for a machine learning algorithm designed to predict tertiary structure. This algorithm can comprise transformer and structure modules described in earlier examples. The parameters of the pretrained ML algorithm that makes predictions on chemical mapping data can be kept fixed or be tuned as part of the training of the algorithm that predicts tertiary structure from sequence.

In some embodiments, the tertiary structure representation comprises a different (sub)set of atoms. The main frame representation may comprise different atoms and not be identical for each nucleotide in the RNA molecule. It may range from zero atoms to a complete list of atoms at the level of individual nucleotides. The structure representation may be without an explicit embedding in terms of 3D coordinates at the level of distance maps that are represented as part of the pairwise features in the structure module and upon which the chemical module then operates. This distance map representation may be augmented with angles.

In some embodiments, the training set may further comprise sequences with data on their secondary structure. This data may be used in various forms, such as in the form of base-pair-probability matrices, either as pairwise input features to the algorithm or as an additional prediction target (as an example, analog to the case of the 2D chemical mapping data described). In the case of the embodiment as an additional prediction, the secondary structure data may or may not be used to optimize the structure module during training (and as such improve tertiary structure prediction capabilities).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A computer-implemented method of predicting a tertiary structure of an RNA molecule of interest comprising:
creating a training data set comprising:
chemical mapping data for a first plurality of RNA molecules, and
tertiary structure data for a second plurality of RNA molecules;
training a machine learning algorithm using the training data set;
applying the trained machine learning algorithm to predict the tertiary structure of the RNA molecule of interest; and
outputting the predicted tertiary structure of the RNA molecule of interest.

2. A computer-implemented method of predicting a tertiary structure of an RNA molecule of interest comprising:
(a) obtaining a machine-learning model, wherein the machine-learning model was trained by a process including:
(i) creating a training data set comprising chemical mapping data for a first plurality of RNA molecules and tertiary structure data for a second plurality of RNA molecules; and
(ii) training the machine-learning model using the training data set;
(b) applying the machine-learning model to predict the tertiary structure of the RNA molecule of interest; and
(c) outputting the predicted tertiary structure of the RNA molecule of interest.

3. The method of claim 2, wherein the chemical mapping data is generated by a process comprising contacting the RNA molecule with a chemical probing agent, optionally wherein the RNA molecule is at least one of the first plurality of RNA molecules or the RNA molecule of interest.

4. The method of claim 3, wherein the chemical probing agent comprises dimethyl sulfate (DMS).

5. The method of claim 3, wherein the chemical probing agent comprises a SHAPE (selective 2′-hydroxyl acylation and primer extension) reagent.

6. The method of claim 5, wherein the SHAPE reagent is 1-methyl-7-nitroisatoic anhydride (1M7), 1-methyl-6-nitroisatoic anhydride (1M6), 5-nitroisatoic anhydride (5NIA), or N-methyl-nitroisatoic anhydride (NMIA).

7. The method of claim 3, wherein the chemical probing agent comprises 2A3 ((2-Aminopyridin-3-yl)(1H-imidazol-1-yl)methanone).

8. The method of claim 2, wherein the RNA molecule of interest comprises a part of a transcriptome.

9. The method of claim 8, wherein the transcriptome is a human transcriptome.

10. The method of claim 2, wherein the training data set comprises chemical mapping data for at least about 10, 100, 500, 1,000, 10,000, 100,000, 1,000,000, or 10,000,000 sequences.

11. (canceled)

12. The method of claim 2, wherein the chemical mapping data is for sequences which occur in different abundance than in natural systems.

13. The method of claim 2, wherein the chemical mapping data was collected from in vitro sources.

14. The method of claim 2, further comprising, before applying the machine-learning model, tuning the machine learning algorithm based on a chemical mapping data of the RNA molecule of interest.

15. The method of claim 2, wherein the machine learning algorithm comprises one or more artificial neural networks (ANNs).

16. The method of claim 15, further comprising training the ANN to predict chemical mapping data for the RNA molecule of interest.

17. The method of claim 16, further comprising predicting the chemical mapping data for the RNA molecule of interest using the predicted tertiary structure of the RNA molecule of interest.

18. The method of claim 16, further comprising predicting the tertiary structure of the RNA molecule of interest using the chemical mapping data for the RNA molecule of interest.

19. The method of claim 16, further comprising predicting the chemical mapping data for the RNA molecule of interest and the predicted tertiary structure of the RNA molecule of interest using embeddings from the same ANN.

20. The method of claim 2, wherein the tertiary structure comprises 3-D coordinates of a plurality of atoms that compose the RNA molecule of interest.

21. The method of claim 20, wherein the tertiary structure comprises 3-D coordinates of each atom that composes the RNA molecule of interest.

22. The method of claim 2, wherein the tertiary structure comprises one or more 3-D coordinates for a plurality of nucleotides that compose the RNA molecule of interest.

23. The method of claim 22, wherein the tertiary structure comprises one or more 3-D coordinates for each nucleotide that composes the RNA molecule of interest.

24. The method of claim 2, wherein the tertiary structure of the RNA molecule of interest is parametrized based on a distance map.

25. The method of claim 2, wherein the tertiary structure of the RNA molecule of interest is parametrized based on a distance map and angles.

26. The method of claim 2, wherein the method does not require determining or predicting a secondary structure of the RNA molecule of interest.

27. The method of claim 2, wherein the method predicts aspects of a tertiary structure of the target RNA that are not captured by a base-pairing prediction of the target RNA.

28. The method of claim 2, wherein the predicted tertiary structure comprises one or more of a pseudoknot, multi-way junction, coaxial stack, a-minor motif, kissing stem-loop, ribose zipper, or tetraloop/tetraloop receptor.

29. The method of claim 2, wherein the chemical mapping data comprises multidimensional chemical mapping data for one or more RNA molecules of the first plurality of RNA molecules.

30. The method of claim 2, wherein the predicted tertiary structure is a target for a pharmaceutical drug.

31. The method of claim 2, further comprising determining a target region or subsequence of the RNA molecule of interest, for a pharmaceutical drug to target, based on the predicted tertiary structure.

32. The method of claim 2, further comprising formulating a pharmaceutical drug based on the predicted tertiary structure.

33. The method of claim 2, wherein the training data set further comprises a multiple sequence alignment of a third plurality of RNA molecules.

34. The method of claim 33, wherein the first plurality of RNA molecules and the second plurality of RNA molecules are different.

35. (canceled)

36. The method of claim 2, wherein the first plurality of RNA molecules are unrelated to the RNA molecule of interest.

37. The method of claim 2, wherein the second plurality of RNA molecules are unrelated to the RNA molecule of interest.

38. The method of claim 33, wherein the third plurality of RNA molecules are unrelated to the RNA molecule of interest.

39. The method of claim 2, wherein an RNA molecule of the first plurality of RNA molecules has no more than about 80%, 70%, 60%, 50%, 40%, 30%, 20%, or less sequence identity to the RNA molecule of interest.

40. A computer-implemented system for predicting a tertiary structure of an RNA molecule of interest comprising a computing device comprising at least one processor and instructions executable by the at least one processor to perform operations comprising:
a) creating a training data set comprising one or more of:
i) chemical mapping data for a first plurality of RNA molecules,
ii) tertiary structure data for a second plurality of RNA molecules;
b) training a machine learning algorithm using the training data set;
c) applying the trained machine learning algorithm to predict the tertiary structure of the RNA molecule of interest; and
d) outputting the predicted tertiary structure of the RNA molecule of interest.

41. One or more non-transitory computer-readable storage media encoded with instructions executable by one or more processors to provide an application for predicting a tertiary structure of an RNA molecule of interest, the application comprising:
a) a training data set module configured to create a training data set comprising: chemical mapping data for a first plurality of RNA molecules, and tertiary structure data for a second plurality of RNA molecules;
b) a training module configured to train a machine learning algorithm using the training data set;
c) an inference module configured to apply the trained machine learning algorithm to predict the tertiary structure of the RNA molecule of interest; and
d) an output module configured to report the predicted tertiary structure of the RNA molecule of interest.

42. One or more non-transitory computer-readable medium comprising:
accessing an RNA tertiary structure prediction system that was manufactured by a process comprising:
creating a training data set comprising chemical mapping data for a first plurality of RNA molecules and tertiary structure data for a second plurality of RNA molecules;
training a machine-learning model using the training data set; and
storing the trained machine-learning model on the non-transitory computer-readable medium; and
computer program code that, when executed by a computing system, cause the computing system to perform operations including:
using the RNA tertiary structure prediction system to predict the tertiary structure of the RNA molecule of interest; and
outputting the predicted tertiary structure of the RNA molecule of interest.

43. A computer-implemented method of predicting a tertiary structure of an RNA molecule of interest comprising:
(a) sending a query for predicting the tertiary structure of the RNA molecule of interest to a computer comprising a machine-learning model, wherein the machine learning algorithm generates the tertiary structure, wherein the machine-learning model was trained by a process including:
(i) creating a training data set comprising chemical mapping data for a first plurality of RNA molecules and tertiary structure data for a second plurality of RNA molecules; and
(ii) training the machine-learning model using the training data set;
(b) receiving the predicted tertiary structure of the RNA molecule of interest from the computer.

44. The method of claim 18, wherein the chemical mapping data for the RNA molecule of interest is one or more of:
a) multidimensional, and
b) originated from chemical probing experiments different from those used to originate the chemical mapping data for a first plurality of RNA molecules.
